(19) United States
(12) Patent Application Publication
(10) Pub. No.: US 2017/0288228 A1
(43) Pub. Date: Oct. 5, 2017

(54) ELECTRODE ACTIVE MATERIAL COMPRISING QUINODIMETHANE DERIVATIVES, AND DERIVATIVES, AND SECONDARY BATTERY COMRISING THE SAME

(71) Applicants: SAMSUNG ELECTRONICS CO., LTD., Suwon-si (KR); MIE UNIVERSITY, Mie (JP)

(72) Inventors: Seitaro ITO, Kanagawa (JP); Tomonobu MIZUMO, Kanagawa (JP); Yuichi AIHARA, Kanagawa (JP); Takahito ITOH, Tsu City (JP); Takahiro UNO, Tsu City (JP)

(21) Appl. No.: 15/460,711

(22) Filed: Mar. 16, 2017

(30) Foreign Application Priority Data
Mar. 31, 2016 (JP) 2016-072079
Feb. 13, 2017 (KR) 10-2017-0019491

(51) Int. Cl. H01M 4/60; H01M 10/0525; C07C 255/34; H01M 10/0569; H01M 10/0565; H01M 10/0568; H01M 4/38; H01M 10/0562

(52) U.S. Cl. CPC H01M 4/60; H01M 4/382; H01M 10/0525; H01M 10/0562; H01M 10/0569; H01M 10/0565; H01M 10/0568; C07C 255/34; H01M 2300/0082; H01M 2300/0068; H01M 2300/0034; H01M 2300/0037

(57) ABSTRACT

An electrode active material including at least one selected from a quinodimethane derivative and a lithium salt thereof, wherein the quinodimethane derivative has at least three linearly-condensed aryl groups and at least two quinodimethane moieties.

### CROSS-REFERENCE TO RELATED APPLICATION

This application claims priority to Japanese Patent Application No. 2016-072079, filed on Mar.31, 2016, in the Japan Patent Office, and Korean Patent Application No. 10-2017-0019491, filed on Feb. 13, 2017, in the Korean Intellectual Property Office, and all the benefits accruing therefrom under 35 U.S.C. §119, the contents of which are incorporated herein in their entirety by reference.

### BACKGROUND

### 1. Field

The present disclosure relates to an electrode active material including a quinodimethane derivative, and a secondary battery including the electrode active material.

### 2. Description of the Related Art

It has recently been found that a high energy density and a long lifespan are required for lithium batteries to be used in electronic devices and electric vehicles. Lithium ion secondary batteries are secondary batteries are widely used because they have such characteristics.

As a type of lithium ion battery, organic secondary batteries in which an organic compound is used as an electrode active material have been objects of active research. Since an organic secondary battery uses an organic material instead of a metal salt such as lithium cobalt oxide as a positive active material, it is expected that a preparation cost and weight thereof will be reduced. Furthermore, through molecular engineering of organic positive active materials to enable a multi-electron reaction, a battery is expected to have increased capacity.

As such organic positive active materials, for example, electron acceptors such as tetracyanoquinodimethane (TCNQ), are widely known (see JP 1999-003708). JP 2015-122237 discloses p-dicyanobenzoquinonemethide and derivatives thereof as positive active materials.

JP 2010-044882 suggests a polymer compound including a cyanoquinodimethane moiety in its backbone, which may be used to obtain a secondary battery having good high-output cycle characteristics.

Conventional low-molecular weight compounds such as tetracyanoquinodimethane described above are capable of a multi-electron reaction, and thus, provide high capacity. For example, tetracyanoquinodimethane is an active material in which a two-electron reaction occurs at cyano moieties of the compound, and thus, a secondary battery using this compound is expected to have a high capacity (for example, a theoretical capacity of 260 milliampere hours per gram “mAh/g”). Such a conventional organic electrode active material has comparatively high output characteristics.

However, in general, an organic electrode active material dissolves easily in an electrolyte solution when it is ionized. For this reason, the organic electrode active material is dissolved in the electrolyte solution during charging, thus inducing an electrochemical reaction with an active material of an opposite electrode on a surface of the opposite electrode, thereby producing a reaction product. This consequently may reduce capacity of the secondary battery and may lead to a loss of the organic electrode active material. Therefore, a secondary battery including a conventional low-molecular weight compound such as tetracyanoquinodimethane may significantly lose its capacity when it undergoes charge and discharge cycles.

JP 2010-044882 discloses a polymer compound which is insoluble in an electrolyte solution due to polymerization. However, polymerization requires introducing an unnecessary structure (such as a backbone, side chains, or the like) into an organic electrode active material, leading to an increased weight of the battery, and its reduced capacity per weight. Accordingly, it is not possible to sufficiently increase capacity of a secondary battery using the polymer compound as a positive active material.

Therefore, there is an increasing demand for an electrode active material for a secondary battery having high capacity and good cycle characteristics.

### SUMMARY

Provided are an electrode active material which may exhibit improved capacity and improved cycle characteristics, and a secondary battery with improved capacity and improved cycle characteristics, which includes the electrode active material.

Additional aspects will be set forth in part in the description which follows and, in part, will be apparent from the description, or may be learned by practice of the presented embodiments.

According to an aspect of an embodiment, an electrode active material includes:

at least one selected from a quinodimethane compound and a lithium salt thereof,

wherein the quinodimethane compound has at least three linearly-condensed aryl groups and at least two quinodimethane moieties.

According to an aspect of another embodiment, a secondary battery includes:

a positive electrode including the above-described electrode active material;

a negative electrode; and

an electrolyte membrane.

### BRIEF DESCRIPTION OF THE DRAWINGS

These and/or other aspects will become apparent and more readily appreciated from the following description of the embodiments, taken in conjunction with the accompanying drawings in which:

FIG. 1 is a schematic view illustrating a structure of a secondary battery according to an embodiment;

FIG. 2 is a charge and discharge curve of a secondary battery prepared according to Example 1;

FIG. 3 is a charge and discharge curve of a secondary battery prepared according to Comparative Example 1; and

FIG. 4 is a graph of capacity retention with respect to number of cycles in the secondary batteries prepared according to Example 1 and Comparative Example 1.

### DETAILED DESCRIPTION

Reference will now be made in detail to embodiments, examples of which are illustrated in the accompanying drawings, wherein like reference numerals refer to like elements throughout, and thus, redundant description thereof will be omitted. In this regard, the present embodiments may have different forms and should not be construed as being limited to the descriptions set forth herein. Accordingly, the embodiments are merely described below, by referring to the figures, to explain aspects of the present disclosure. As used herein, the term “and/or” includes any and all combinations of one or more of the associated listed items. Expressions such as “at least one selected from,” when preceding a list of elements, modify the entire list of elements and do not modify the individual elements of the list.

It will be understood that when an element is referred to as being “on” another element, it can be directly in contact with the other element or intervening elements may be present therebetween. In contrast, when an element is referred to as being “directly on” another element, there are no intervening elements present.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

The terminology used herein is for the purpose of describing particular embodiments only and is not intended to be limiting. As used herein, the singular forms “a,” “an,” and “the” are intended to include the plural forms as well, unless the context clearly indicates otherwise.

The term “or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10%, 5% of the stated value.

Electrode active material and quinodimethane derivatives

Prior to description of secondary batteries according to embodiments, electrode active materials and quinodimethane derivatives (compounds) according to embodiments will now be described.

According to an aspect of the present disclosure, an electrode active material includes at least one selected from a quinodimethane derivative and a lithium salt thereof, wherein the quinodimethane derivative has at least three linearly-condensed aryl groups and at least two quinodimethane moieties.

A quinodimethane derivative with a quinodimethane moiety in such a straight-chain (linear) acene aromatic hydrocarbon may have a comparatively low solubility in an electrolyte. Accordingly, when the electrode active material according to an embodiment includes such a quinodimethane derivative, dissolution of the electrode active material into an electrolyte may be suppressed during a charge and discharge cycle of a secondary battery.

Theoretically carbon atoms from methine groups in the quinodimethane moiety may become carbanions by accepting electrons during charging, and at the same time, form a salt with cations such as lithium ions. Such carbanions that have accepted electrons are stabilized in such a composite structure including the quinodimethane moiety. Each quinodimethane moiety has two methine groups. Accordingly, the quinodimethane derivative having two quinodimethane moiety may theoretically undergo a multi-electron reaction involving 4 or more electrons, and may have a comparatively high capacity per weight. Consequently, a secondary battery including such a quinodimethane derivative as an electrode active material may have high capacity.

The quinodimethane derivative also has a polycyclic aromatic hydrocarbon as a major backbone, which is highly planar, and thus, may relatively improve the capacity density per volume of the electrode active material.

Due to the above-described reasons, when an electrode active material according to an embodiment including a quinodimethane derivative such as that described above is used, a secondary battery with improved capacity and improved cycle characteristics, as compared with the one including a conventional compound, may be provided.

In some embodiments, the quinodimethane derivative of the electrode active material is not specifically limited, and may be, for example, a compound represented by Formula 1:

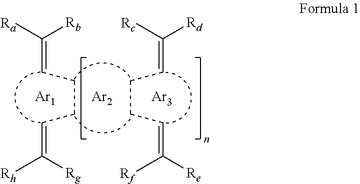

In Formula 1,

Ar₁ to Ar₃ may each independently be a substituted or unsubstituted C₅-C₆₀ carbocyclic group, or a substituted or unsubstituted C₁-C₆₀ heterocyclic group;

Rₐ to Rₕ may each independently be selected from a hydrogen atom, a halogen atom, a hydroxyl group, a nitro group, a cyano group, a carboxylic acid group, a substituted or unsubstituted C₁-C₆₀ alkyl group, a substituted or unsubstituted C₆-C₆₀ aryl group, and a substituted or unsubstituted C₁-C₆₀ heteroaryl group;

n may be an integer of 1 or greater;

at least one substituent of each of the substituted C₅-C₆₀ carbocyclic group and substituted C₁-C₆₀ heterocyclic group is selected from

deuterium (-D), —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from a deuterium, —F, —Cl, —Br, —I, a hydroxyl group, a cyano group, a nitro group, an amidino group, a hydrazino group, a hydrazono group, a C₃-C₁₀ cycloalkyl group, a C₁-C₁₀ heterocycloalkyl group, a C₃-C₁₀ cycloalkenyl group, a C₁-C₁₀heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₁₁)(Q₁₂)(Q₁₃), —N(Q₁₁)(Q₁₂), -B(Q₁₁)(Q₁₂), —C(═O)(Q₁₁), —S(═O)₂(Q₁₁), and —P(═O)(Q₁₁)(Q₁₂);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₂₁)(Q₂₂)(Q₂₃), —N(Q₂₁)(Q₂₂), —B(Q₂₁)(Q₂₂), —C(-0)(Q₂₁), —S(═O)₂(Q₂₁), and —P(═O)(Q₂₁)(Q₂₂);

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), and —P(═O)(Q₃₁)(Q₃₂),

wherein Q₁₁ to Q₁₃, Q₂₁ to Q₂₃, and Q₃₁ to Q₃₃ may each independently be selected from hydrogen, deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, a monovalent non-aromatic condensed heteropolycyclic group, a biphenyl group, and a terphenyl group.

In some embodiments, at least one selected from Rₐ to Rₕ may be a cyano group. For example, each of two or more of Rₐ to Rₕ may be a cyano group. However, embodiments are not limited thereto.

In some embodiments, Ar₁ to Ar₃ may each independently be selected from:

a benzene group, a naphthalene group, a phenalene group, a phenanthrene group, an anthracene group, a fluoranthene group, a triphenylene group, a pyrene group, a chrysene group, a naphthacene group, a picene group, a perylene group, and a pentaphene group; and

a benzene group, a naphthalene group, a phenalene group, a phenanthrene group, an anthracene group, a fluoranthene group, a triphenylene group, a pyrene group, a chrysene group, naphthacene group, a picene group, a perylene group, and a pentaphene group, each substituted with at least one selected from a deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), and —P(═O)(Q₃₁)(Q₃₂),

wherein Q₃₁ to Q₃₃ may each independently be selected from a C₁-C₁₀ alkyl group, a C₁-C₁₀ alkoxy group, a phenyl group, a biphenyl group, a terphenyl group, and a naphthyl group.

For example, the quinodimethane derivative may be a compound represented by Formula 1A or 1B. However, embodiments are not limited thereto:

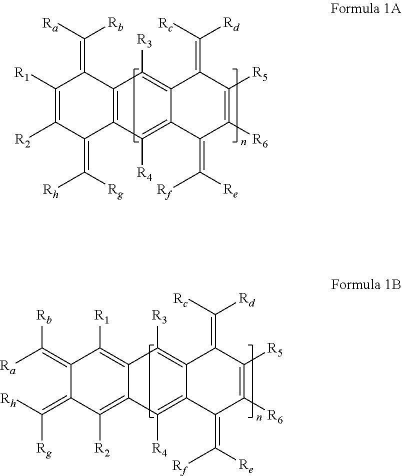

In Formulae 1A and 1B,

Rₐ to Rₕ may each independently be selected from a hydrogen atom, a halogen atom, a hydroxyl group, a nitro group, a cyano group, a carboxylic acid group, a substituted or unsubstituted C₁-C₆₀ alkyl group, a substituted or unsubstituted C₆-C₆₀ aryl group, and a substituted or unsubstituted C₁-C₆₀ heteroaryl group,

at least one selected from Rₐ to Rₕ may be a cyano group,

R₁ to R₆ may each independently be selected from a hydrogen atom, a halogen atom, a hydroxyl group, a nitro group, a cyano group, a carboxylic acid group, a substituted or unsubstituted C₁-C₆₀ alkyl group, a substituted or unsubstituted C₆-C₆₀ aryl group, and a substituted or unsubstituted C₁-C₆₀ heteroaryl group, and

n may be an integer of 1 or greater.

In some embodiments, the quinodimethane derivative may be a compound represented by Formula 1A-1:

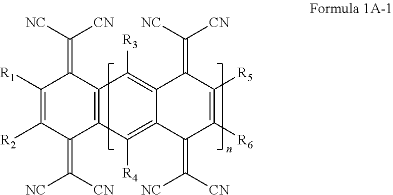

In Formula 1A-1, R₁ to R₆ may each independently be selected from a hydrogen atom, a halogen atom, a hydroxyl group, a nitro group, a cyano group, a carboxylic acid group, a substituted or unsubstituted C₁-C₆₀ alkyl group, a substituted or unsubstituted C₆-C₆₀ aryl group, and a substituted or unsubstituted C₁-C₆₀ heteroaryl group, and

n may be an integer of 1 or greater.

In Formula 1A-1, the alkyl group of R₁ to R₆ may be, for example, a C1-C20 linear or branched alkyl group. For example, the alkyl group may be a linear alkyl group, such as a methyl group, an ethyl group, an n-propyl group, an n-butyl group, a n-pentyl group, an n-hexyl group, an n-heptyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, an n-nonadecyl group, or an n-icosyl group; or a branched alkyl group, such as an iso-propyl group, an iso-butyl group, a sec-butyl group, a tert-butyl group, a 1-methylbutyl group, a 2-methylbutyl group, a 3-methylbutyl group, a 1,1-dimethylpropyl group, a 1,2-dimethylpropyl group, a 1-ethylpropyl group, a 1-methylpentyl group, a 2-methylpentyl group, a 3-methylpentyl group, a 4-methylpentyl group, a 1,1-dimethylbutyl group, a 1,2-dimethylbutyl group, a 2,2-dimethylbutyl group, a 2,3-dimethylbutyl group, a 3,3-dimethylbutyl group, a 1-methyl-1-ethylpropyl group, a 2-methyl-1-ethylpropyl group, a 1-methyl-2-ethylpropyl group, a 1,1,2-trimethylpropyl group, or a 1,2,2-trimethylpropyl group. The number of carbon atoms in such a linear or branched alkyl group is not specifically limited, and may be, for example, 1 to 20, and in some embodiments, may be 1 to 10, and in some other embodiments, 1 to 5, and in still other embodiments, 1 to 3.

The aryl group of R₁ to R₆ is not specifically limited. For example, the aryl group may be a monocyclic aromatic group, such as a phenyl group; a non-condensed polycyclic aromatic group, such as a biphenyl group, a terphenyl group, a quaterphenyl group, a quinquephenyl group, a sexiphenyl group, a fluoroanthenyl group, or a triphenylenyl group; or a condensed polycyclic aromatic group, such as a naphtyl group, an anthryl group, a phenanthryl group, a fluorenyl group, an indenyl group, a pyrenyl group, an acetonaphtenyl group, or a bis-phenylfluorenyl group. The number of carbon atoms in the aryl ring of R₁ to R₆ is not specifically limited, and may be, for example, 6 to 24, and in some embodiments, may be 6 to 12.

The heteroaryl group of R₁ to R₆ may be, for example, a monocyclic heteroaryl group, such as a pyrrolyl group, an imidazole group, a pyrazolyl group, an oxazolyl group, an isoxazolyl group, an oxadiazolyl group, a thiazolyl group, a furanyl group, a pyranyl group, a thienyl group, a pyridyl group, a pyrazyl group, a pyrimidinyl group, a pyridazinyl group, a triazinyl group, a quinolyl group, or an isoquinolyl group; or a polycyclic heteroaryl group, such as a benzo(pyridyl)furanyl group, a benzofuranyl group, a benzothienyl group, an indolyl group, a carbazolyl group, a carbolinyl group, a phenanthridinyl group, an acridinyl group, a perimidinyl group, a phenanthrolinyl group, a benzoxazolyl group, a benzothiazolyl group, a quinoxalyl group, a benzimidazolyl group, a pyrazolyl group, a dibenzofuranyl group, or a dibenzothienyl group. The number of carbon atoms in the heteroaryl ring of R₁ to R₆ is not specifically limited, and may be, for example, 5 to 24, and in some embodiments, 5 to 12.

Substituents of the alkyl group, the aryl group, and the heteroaryl group are not specifically limited. Non-limiting examples of substituents of the alkyl group, the aryl group, and the heteroaryl group are a cyano group, a silyl group, a C1-C10 mono-, di-, or tri-alkylsilyl group, a C1-C10 linear, branched or cyclic alkyl group, a C1-C10 linear or branched alkoxy group, a C6-C15 aryl group, a C6-C15 aryloxy group, a C6-C15 arylcarbonyl group, a C3-C32 heterocyclyl group, a C1-C10 mono- or di-alkyl amino group, and a C6-C15 mono- or di-aryl amino group. Unless stated otherwise, the term simply “substituted” may be construed as meaning that a group is substituted with such substituents listed above.

The halogen atom of R₁ to R₆ may be a fluorine atom, a chlorine atom, a bromine atom, or an iodine atom.

For example, R₁ to R₆ in Formula 1A-1 may each independently be a hydrogen atom, a halogen atom, or a C1-05 linear or branched alkyl group. In some embodiments, R₁ to R₆ may each independently be a hydrogen atom, a fluorine atom, a chlorine atom, a methyl group, an ethyl group, or an n-propyl group, and in some other embodiments, may each independently be a hydrogen atom or a methyl group, and in still other embodiments, may be a hydrogen atom.

In some embodiments, R₁ to R₆ may be the same as or differ from one another. For example, R₁ to R₆ may be the same.

In Formula 1A-1, n may be any integer of 1 or greater. In some embodiments, n may be selected from 1 to 4, and in some other embodiments, may be 1.

According to the above-described mechanism, each carbon atom of the dicyanomethine groups of the quinodimethane derivative of Formula 1A-1 may become an anion by accepting electrons while at the same time forming a salt with cations such as lithium ions. The quinodimethane derivative may have multiple oxidation-reduction potentials (ORPs) because it may be involved in multiple oxidation and reduction reactions.

In some embodiments, the quinodimethane derivative may have two or more oxidation-reduction potentials (ORPs) within a voltage range of about 1.0 volts (V) to about 4.0 V with respect to Li/Li⁺, and in some embodiments, may have two or more oxidation-reduction potentials (ORPs) within a voltage range of about 2.0 V to about 3.5 V with respect to Li/Li⁺. Since the quinodimethane derivative has oxidation-reduction potentials (ORPs) within these voltage ranges, the quinodimethane derivative may generate sufficient energy through the oxidation-reduction reactions and may prevent deterioration of an electrolyte. Due to having two or more oxidation-reduction potentials (ORPs) within the above voltage ranges, the quinodimethane derivative may sufficiently enhance the capacity of a secondary battery including the same.

In some embodiments, the electrode active material may include only one or more of the quinodimethane derivatives described above, for example, one or more of the quinodimethane derivatives represented by Formula 1A-1. In some other embodiments, the electrode active material may include only one of the quinodimethane derivatives described above, for example, one of the quinodimethane derivatives represented by Formula 1A-1.

In some embodiments, the electrode active material may include the quinodimethane derivative as described above, for example, the quinodimethane derivatives represented by Formula 1A-1 as, for example, a major component. In some embodiments, the electrode active material may include about 60 percent by weight (wt %) or more, and in some other embodiments, about 90 wt % or more, and in still other embodiments, about 95 wt % or more of a quinodimethane derivative as described above, for example, a quinodimethane derivative represented by Formula 1A-1, based on a total weight of the electrode active material.

In some other embodiments, the electrode active material may substantially include a quinodimethane derivative as described above, for example, a quinodimethane derivative represented by Formula 1A-1, and in still other embodiments, may consist of only a quinodimethane derivative as described above, for example, a quinodimethane derivative represented by Formula 1A-1. Accordingly, a secondary battery including an electrode active material according to any of the above-described embodiments may have improved capacity and improved cycle characteristics.

In some embodiments, the quinodimethane derivatives may be synthesized using a known synthesis method.

In some embodiments, the electrode active material may further include, in addition to the quinodimethane derivative as described above, an additional electrode active material. The additional electrode active material may be, for example, one of various commonly known positive active materials and negative active materials, which may be organic or inorganic.

The positive active material may be, for example, a lithium-containing transition metal oxide or solid solution oxide, as a material capable of electrochemical intercalation and deintercalation of lithium ions. For example, the lithium-containing transition metal oxide may be a Li—Co composite oxide such as LiCoO₂, a Li—Ni—Co—Mn composite oxide such as LiNiₓCo_{y}Mn_{z}O₂, a Li—Ni composite oxide such as LiNiO₂, or a Li—Mn composite oxide such as LiMn₂O₄. The solid solution oxide may be, for example, LiₐMnₓCo_{y}Ni_{z}O₂ (wherein 1.150≦a≦1.430, 0.45≦x≦0.6, 0.10≦y≦0.15, and 0.20≦z≦0.28), LiMnₓCo_{y}Ni_{z}O₂ (wherein 0.3≦x≦0.85, 0.10≦y≦0.3, and 0.10≦z≦0.3), or LiMn_{1.5}Ni_{0.5}O₄.

The negative active material may be, for example, a graphite active material (for example, artificial graphite, natural graphite, a mixture of artificial graphite and natural graphite, natural graphite coated with artificial graphite, or the like), silicon oxide or tin oxide particles, a silicon (Si)- or tin (Sn)-based alloy, a mixture of silicon oxide or tin oxide particles and a graphite active material, or a titanium oxide (TiOₓ) such as Li₄Ti₅O₁₂, wherein the silicon oxide may be represented by the formula of SiOₓ(wherein 0≦x≦2). However, embodiments of the present disclosure are not limited thereto. For example, the negative active material may be an alkali metal, such as sodium metal, lithium metal, or the like.

In some embodiments, the electrode active material may be used as a positive active material, and the electrode active material may include an additional positive active material, in addition to the quinodimethane derivative.

In some embodiments, the electrode active material may include a relatively small amount of the additional electrode active material, in addition to the quinodimethane derivative. In some embodiments, when an electrode active material according to any of the embodiments includes an additional electrode active material, in addition to the quinodimethane derivative, the amount of the additional electrode active material may be about 40 wt % or less, and in some embodiments, about 10 wt % or less, and in some other embodiments, about 5 wt % or less, based on a total weight of the electrode active material.

An electrode active material according to any of the above-described embodiments may be solely a positive active material or a negative active material. In some embodiments, the electrode active material may be a positive active material in order to make the best use of the electron acceptability of the quinodimethane moiety.

Structure of Secondary Battery

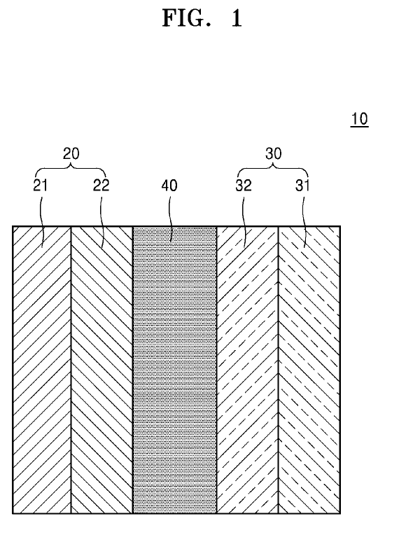

Hereinafter, a detailed structure of a secondary battery according to an embodiment will now be described with reference to FIG. 1. FIG. 1 is a schematic view illustrating a structure of a secondary battery 10 according to an embodiment.

Referring to FIG. 1, the secondary battery 10 may include a positive electrode 20, a negative electrode 30, and an electrolyte membrane 40. The shape of the secondary battery 10 is not particularly limited, and may be any shape, for example, a cylindrical, rectangular, laminate, or button shape.

The positive electrode 20 may include a current collector 21 and a positive active material layer 22. The current collector 21 may be any electric conductor, for example, aluminum (Al), a stainless steel, or a nickel-plated steel.

The positive active material layer 22 may at least include a positive active material and a conducting agent. The positive active material layer 22 may further include a binder. The amounts of the positive active material, the conducting agent and the binder are not specifically limited, and may be the same as amounts commonly used in secondary batteries.

The positive active material may be an electrode active material according to any of the above-described embodiments.

The conducting agent may be, for example, a carbon black such as ketjen black or acetylene black; fibrous carbon such as natural graphite, artificial graphite, carbon nanotubes, graphene, and carbon nanofibers; a composite of such fibrous carbon and carbon black; or a conductive polymer. However, the conducting agent is not specifically limited, and may be any material able to increase conductivity of the positive electrode.

The binder may be, for example, a fluorine-based resin such as polyvinylidene difluoride, fluoroelastomer, or polytetrafluoroethylene; a thermoplastic resin such as polyethylene or polypropylene; an ethylene-propylene-diene terpolymer; a styrene-butadiene rubber; an acrylonitrile-butadiene rubber; polyvinyl acetate, polymethylmethacrylate; or a nitrocellulose. However, the binder is not specifically limited thereto, and may be any material with the ability to bind the positive active material and the conducting agent to the current collector 21 while having oxidation resistance, durability against a high potential of the positive electrode, and stability in an electrolyte solution.

The positive active material layer 22 may be formed, for example, by dispersing a positive active material, a conducting agent, and a binder as described above in an appropriate organic solvent (for example, N-methyl-2-pyrrolidone) to prepare a positive electrode slurry, and coating the positive electrode slurry on the current collector 21, followed by drying and roll-pressing a product resulting from the coating. The thickness of the positive active material layer 22 after the roll-pressing is not specifically limited, and may be any thickness that a positive active material layer of a conventional secondary battery may have.

The negative electrode 30 may include a current collector 31 and a negative active material layer 32. The current collector 31 may be any electric conductor, for example, copper (Cu), a Cu alloy, aluminum (Al), a stainless steel, or a nickel-plated steel.

The negative active material layer 32 may include any material available in a negative active material layer of a secondary battery. For example, the negative active material layer 32 may include a negative active material, and may optionally further include a binder. For example, the negative active material may be an alkali metal, and in some embodiments, may be lithium metal or sodium metal, and in some other embodiments, may be lithium metal.

The binder is not specifically limited, and may be any known in the art binder. The binder may be the same binder as that used in the positive electrode. A mass ratio of the negative active material to the binder is not specifically limited, and may be the same as a mass ratio commonly applied to conventional secondary batteries.

When the negative active material is not an alkali metal, the negative active material layer 32 may be formed, for example, by dispersing the negative active material and a binder in an appropriate solvent (for example, water) to prepare a negative electrode slurry and coating the negative electrode slurry on the current collector 31, followed by drying and roll-pressing a product resulting from the coating. The thickness of the negative active material layer 32 is not specifically limited, and may be any thickness that a negative active material layer of a conventional secondary battery may have. When the negative active material is an alkali metal, the negative active material layer 32 may be an alkali metal foil.

The electrolyte membrane 40 may include a solid electrolyte, a polymer electrolyte, or a non-aqueous electrolyte solution. When the electrolyte membrane 40 includes a solid electrolyte or a polymer electrolyte, dissolution of the positive active material contacting the electrolyte may be prevented. In some embodiments, the electrolyte membrane 40 may include a solid electrolyte, for example, a phosphoric acid solid electrolyte or a sulfide solid electrolyte, in view of charge and discharge characteristics, cycle characteristics, and stability. Even when the electrolyte membrane 40 includes a non-aqueous electrolyte solution, dissolution of an electrode active material according to any of the above-described embodiments may be sufficiently inhibited when in contact with the non-aqueous electrolyte solution.

The phosphoric acid solid electrolyte may include, for example, Li₃N, LISICON, LIPON (Li_{3+y}PO₄₋ₓNₓ), thio-LIS ICON (Li_{3.25}Ge_{0.25}P_{0.75}S₄), or Li₂O—Al₂O₃—TiO₂—P₂SO₅ (LATP). The sulfide solid electrolyte may be a composite of Li₂S and P₂S₅ in a ratio of 80:20, which may have good ion conductivity. For example, the composite of Li₂S and P₂S₅ may also include Li₇PS₆, Li₄P₂S₆, Li₃PS₄, or Li₇P₃S₁₁.

The polymer electrolyte may be prepared by curing a polymer precursor. The polymer precursor may be a polymer-forming monomer, for example, acrylate or methacrylate. Such a polymer-forming monomer may be used in combination with a gel-forming compound, such as polyethylene oxide (PEO), polypropylene oxide (PPO), polyacrylonitrile (PAN), polyvinylidene fluoride (PVDF), polymethacrylate (PMA), polymethylmethacrylate (PMMA), or a copolymer thereof.

In curing the polymer precursor, a polymerization initiator may be used. The polymerization initiator may be an organic peroxide or an azo compound. Examples of the organic peroxide are diacylperoxides such as diacetyl peroxide, dibenzoyl peroxide, dilauroyl peroxide, and bis(3,5,5-trimethylhexanoyl)peroxide; peroxydicarbonates such as di(4-tert-butylcyclohexyl) peroxydicarbonate, di-(2-ethylhexyl) peroxydicarbonate, di-iso-propyl peroxydicarbonate, di-3-methoxybutyl peroxydicarbonate, t-butylperoxy-iso-propylcarbonate, t-butylperoxy-2-ethylhexylcarbonate, 1,6-bis(t-butylperoxycarbonyloxy)hexane, and diethylene glycol-(t-butylperoxycarbonate); or peroxy esters such as t-butyl peroxypivalate, t-amylperoxypivalate, t-butyl peroxy-2-ethyl-hexanoate, t-hexylperoxypivalate, t-butylperoxyneoheptanoate, t-hexylperoxypivalate, 1,1,3,3-tetramethylbutylperoxyneodecarbonate, 1,1,3,3-tetramethylbutyl-2-ethyl hexanoate, t-amylperoxy-2-ethylhexanoate, t-butylperoxyiso-butyrate, t-amylperoxy-3,5,5-trimethylhexanoate, t-butylperoxy-3,5-trimethylhexanoate, t-butylperoxyacetate, t-butylperoxybenzoate, and di-butylperoxytrimethyladipate.

The non-aqueous electrolyte solution may include an electrolyte salt and a solvent. The solvent may be a non-aqueous solvent capable of dissolving the electrolyte salt. Examples of the solvent are cyclic carbonates such as propylene carbonate, ethylene carbonate, butylene carbonate, chloroethylene carbonate, fluoroethylene carbonate, and vinylene carbonate; cyclic carbonate esters such as γ-butyrolactone and γ-valerolactone; linear carbonates such as dimethyl carbonate (DMC), diethyl carbonate, and ethylmethyl carbonate; linear esters such as methyl formate, methyl acetate, and methyl butyrate; linear or cyclic ethers such as tetrahydrofuran and a derivative thereof, 1,3-dioxane, 1,4-dioxane, 1,2-dimethoxyethane, 1,4-dibutoxyethane, 1,1,2,2-tetrafluoroethyl-2,2,3,3-tetrafluoropropyl ether, and methyldiglyme; nitriles such as 3-methoxypropionitrile, acetonitrile, and benzonitrile; and known organic solvents such as dioxolane or a derivative thereof, ethylene sulfide, sulfolane, sultone or a derivative thereof, dimethyl sulfoxide (DMSO), trimethyl phosphate, and triphenyl phosphate. These listed solvents may be used alone or in a combination of at least two of the above solvents. When a combination of at least two of these solvents is used, a mixing ratio of the solvents may be the same as a mixing ratio commonly applied to conventional secondary batteries.

For example, the solvent may be an ionic liquid such as N,N-diethyl-N-ethyl-N-(2-methoxyethyl)ammonium bis(tifluorosulfonyl)imide, N-methyl-N-propylpiperidinium bis(tifluorosulfonyl)imide, N-methyl-N-propylimidazolium bis(tifluorosulfonyl)imide, or 1-ethyl-3-butylimidazolium tetrafluoroborate, which may be used alone or in a combination of at least two of these ionic liquids.

The electrolyte salt (supporting salt) may be an electrolyte such as a lithium salt, a sodium salt, and a potassium salt. The electrolyte salt may be an inorganic ion salt including one of lithium (Li), sodium (Na), and potassium (K), for example, LiClO₄, LiBF₄, LiAsF₆, LiAsF₅, LiPF₆, LiSCN, LiBr, Lil, Li₂SO₄, Li₂B₁₀Cl₁₀, NaClO₄, NaI, NaSCN, NaBr, KClO₄, or KSCN; or an organic ion salt, for example, LiCF₃SO₃, LiN(CF₃SO₂)₂, LiN(FSO₂)₂, LiN(C₂F₅SO₂)₂, LiN(CF₃SO₂)(C₄F₉SO₂), LiC(CF₃SO₂)₃, LiC(C₂F₅SO₂)₃, Li(C₄F₉SO₃), (CH₃)₄NBF₄, (CH₃)₄NBr, (C₂H₅)₄NClO₄, (C₂H₅)₄NI, (C₃H₇)₄NBr, (n-C₄H₉)₄NClO₄, (n-C₄H₉)₄NI, (C₂H₅)₄N-maleate, (C₂H₅)₄N-benzoate, (C₂H₅)₄N-phthalate, lithium stearylsulfate, lithium octylsulfate, or lithium dodecylbenzenesulphonate. These electrolyte salts may be used alone or in a combination of at least two thereof. An excess of these electrolyte salts may be added within a range in which battery characteristics are exhibited. For example, the amount of the electrolyte salt may be about 2 parts to about 70 parts by weight, and in some embodiments, about 20 parts to about 50 parts by weight, based on 100 parts by weight of a total weight of the solvent.

The non-aqueous electrolyte solution may further include an additive, as long as it does not deteriorate electrical characteristics of the electrode active material. For example, the non-aqueous electrolyte solution may further include any of a variety of additives such as an agent capable of forming a negative electrode solid electrolyte interface (SEI) layer, a surfactant, or the like.

Examples of the additive are succinic anhydride, lithium bis(oxalate)borate, lithium tetrafluoroborate, a dinitrile compound, propane sultone, butane sultone, propene sultone, 3-sulfolene, fluorinated arylether, and fluorinated methacrylate. The amount of the additive may be the same as an amount of an additive commonly used in conventional secondary batteries.

The electrolyte membrane 40 or a non-aqueous electrolyte solution may be used by impregnation on a separator. The separator is not specifically limited, and may be any separator available for use in secondary batteries. The separator may be a porous membrane or non-woven fabric having good high-rate discharge performance, or a combination thereof. The separator may be coated with an inorganic material, for example, Al₂O₃, Mg(OH)₂, or SiO₂, or may include such an inorganic material as a filler.

A material used to form the separator may be, for example, a polyolefin resin such as polyethylene or polypropylene, a polyester resin such as polyethylene terephthalate or polybutylene terephthalate, polyvinylidene difluoride, a vinylidene difluoride-hexafluoropropylene copolymer, a vinylidene difluoride-perfluorovinylether copolymer, a vinylidene difluoride-tetrafluoroethylene copolymer, a vinylidene difluoride-trifluoroethylene copolymer, a vinylidene difluoride-fluoroethylene copolymer, a vinylidene difluoride-hexafluoroacetone copolymer, a vinylidene difluoride-ethylene copolymer, a vinylidene difluoride-propylene copolymer, a vinylidene difluoride-trifluoropropylene copolymer, a vinylidene difluoride-tetrafluoroethylene-hexafluoropropylene copolymer, or a vinylidene difluoride-ethylene-tetrafluoroethylene copolymer. Porosity of the separator is not specifically limited, and may be at a level found in a separator of a conventional secondary battery.

In some embodiments, the secondary battery may have a cutoff potential of about 1.8 V to about 3.8 V, and a discharge capacity of about 150 mAhg⁻¹ or greater after 2 to 4 charge and discharge cycles at about 25° C.

In some embodiments, the secondary battery may have a cutoff potential of about 1.8 V to about 3.8 V, and capacity retention of about 80% or greater after 50 charge and discharge cycles at about 25° C.

Method of Manufacturing Secondary Battery

Hereinafter, a method of manufacturing the secondary battery 10 will now be described. However, the secondary battery 10 may be manufactured by any methods used in the art, not limited to the method described below. The secondary battery 10 may be manufactured in the same manner as that used to manufacture a conventional secondary battery, except that an electrode active material according to any of the above-described embodiments is used. A method of manufacturing the secondary battery 10 according to an embodiment is schematically described as follows.

The positive electrode 20 may be manufactured as follows. First, a mixture of a positive active material, a conducting agent, and a binder in an appropriate ratio may be dispersed in an organic solvent (for example, N-methyl-2-pyrrolidone) to prepare a positive electrode slurry. Next, the positive electrode slurry may be applied to the current collector 21 (for example, by coating) and dried to form the positive active material layer 22. The coating method is not specifically limited, and may be chosen from, for example, a method using a knife coater and a method using a gravure coater, in which the coating methods may be substantially the same. The positive active material layer 22 may then be pressed to a desired thickness by using a presser, to thereby manufacture the positive electrode 20. A thickness of the positive active material layer 22 is not specifically limited, and may be any thickness that a positive active material layer of a conventional secondary battery may have.

The negative electrode 30 may be manufactured in the same manner as the positive electrode 20. First, a mixture of a negative active material and a binder in an appropriate ratio may be dispersed in a solvent (for example, water) to prepare a negative electrode slurry. Next, the negative electrode slurry may be applied to the current collector 31 (for example, by coating) and dried to form the negative active material layer 32. The negative active material layer 32 may then be pressed to a desired thickness by using a presser, to thereby manufacture the negative electrode 30. A thickness of the negative active material layer 32 is not specifically limited, and may be any thickness that a negative active material layer of a conventional secondary battery may have. When the negative active material layer 32 is an alkali metal such as lithium, the negative electrode 30 may be manufactured by placing a metal coil and the current collector 31 to overlap with one another.

Next, the separator may be disposed between the positive electrode 20 and the negative electrode 30 to form an electrode assembly with the electrolyte membrane 40 between the positive electrode 20 and the negative electrode 30. Next, the electrode assembly may be shaped into a desired form (for example, a cylindrical, rectangular, laminate, or button type), accommodated in a battery case, followed by injecting an electrolyte solution having a desired composition into the battery case to impregnate pores of the separator with the electrolyte solution, to thereby complete the manufacture of the secondary battery 10.

When a polymer gel electrolyte is used as the electrolyte membrane 40, a polymer gel electrolyte precursor may be injected between the positive electrode 20 and the negative electrode 30 in the battery case, and then cured by thermal treatment, to thereby form the electrolyte membrane 40 consisting of the polymer gel electrolyte. When a solid electrolyte is used as the electrolyte membrane 40, the electrolyte membrane 40 may be prepared using a coating solution prepared by dispersing the solid electrolyte in a polar solvent such as N-methylpyrrolidone (NMP) or dimethylformamide (DMF). For example, the coating solution may be coated on a support and dried to remove the solvent, and a resulting layer may be separated from the support to obtain the electrolyte membrane 40. The electrolyte membrane 40 may also be formed by other methods, for example, by pressure-molding the solid electrolyte and a binder powder.

A C₁-C₆₀ alkyl group as used herein may refer to a linear or branched aliphatic saturated hydrocarbon monovalent group having 1 to 60 carbon atoms, and non-limiting examples thereof include a methyl group, an ethyl group, a propyl group, an iso-butyl group, a sec-butyl group, a tert-butyl group, a pentyl group, an iso-amyl group, and a hexyl group. A C₁-C₆₀ alkylene group as used herein may refer to a divalent group having the same structure as the C₁-C₆₀ alkyl group.

A C₁-C₆₀ alkoxy group as used herein may refer to a monovalent group represented by —OA₁₀₁ (wherein A₁₀₁ is the C₁-C₆₀ alkyl group), and non-limiting examples thereof include a methoxy group, an ethoxy group, and an iso-propoxy group.

A C₂-C₆₀ alkenyl group as used herein may refer to a hydrocarbon group having at least one carbon-carbon double bond in the middle or at either terminal end of the C₂-C₆₀ alkyl group, and non-limiting examples thereof include an ethenyl group, a propenyl group, and a butenyl group. A C₂-C₆₀ alkenylene group as used herein may refer to a divalent group having the same structure as the C₂-C₆₀ alkenyl group.

A C₂-C₆₀ alkynyl group as used herein may refer to a hydrocarbon group having at least one carbon-carbon triple bond in the middle or at either terminal end of the C₂-C₆₀ alkyl group, and non-limiting examples thereof include an ethynyl group and a propynyl group. A C₂-C₆₀ alkynylene group as used herein may refer to a divalent group having the same structure as the C₂-C₆₀ alkynyl group.

A C₃-C₁₀ cycloalkyl group as used herein may refer to a monovalent saturated hydrocarbon monocyclic saturated group having 3 to 10 carbon atoms, and non-limiting examples thereof include a cyclopropyl group, a cyclobutyl group, a cyclopentyl group, a cyclohexyl group, and a cycloheptyl group. A C₃-C₁₀ cycloalkylene group as used herein may refer to a divalent group having the same structure as the C₃-C₁₀ cycloalkyl group.

A C₁-C₁₀ heterocycloalkyl group as used herein may refer to a monovalent saturated monocyclic group having at least one heteroatom selected from N, O, P, Si and S as a ring-forming atom and 1 to 10 carbon atoms, and non-limiting examples thereof include a tetrahydrofuranyl group and a tetrahydrothiophenyl group. A C₁-C₁₀ heterocycloalkylene group as used herein may refer to a divalent group having the same structure as the C₁-C₁₀ heterocycloalkyl group.

A C₃-C₁₀ cycloalkenyl group carbon as used herein may refer to a monovalent monocyclic group that has 3 to 10 carbon atoms and at least one carbon-carbon double bond in the ring thereof and does not have aromaticity, and non-limiting examples thereof include a cyclopentenyl group, a cyclohexenyl group, and a cycloheptenyl group. A C₃-C₁₀ cycloalkenylene group as used herein may refer to a divalent group having the same structure as the C₃-C₁₀ cycloalkenyl group.

A C₁-C₁₀ heterocycloalkenyl group as used herein may refer to a monovalent monocyclic group that has at least one heteroatom selected from N, O, P, Si, and S as a ring-forming atom, 1 to 10 carbon atoms, and at least one double bond in the ring. Non-limiting examples of the C₂-C₁₀ heterocycloalkenyl group include 2,3-dihydrofuranyl group and a 2,3-dihydrothiophenyl group. A C₁-C₁₀ heterocycloalkenylene group as used herein may refer to a divalent group having the same structure as the C₁-C₁₀ heterocycloalkenyl group.

A C₆-C₆₀ aryl group as used herein may refer to a monovalent group having an aromatic system having 6 to 60 carbon atoms, and a C₆-C₆₀ arylene group as used herein may refer to a divalent group having an aromatic system having 6 to 60 carbon atoms. Non-limiting examples of the C₆-C₆₀ aryl group include a phenyl group, a naphthyl group, an anthracenyl group, a phenanthrenyl group, a pyrenyl group, and a chrysenyl group. When the C₆-C₆₀ aryl group and the C₆-C₆₀ arylene group each independently include two or more rings, the respective rings may be fused to each other or may be linked with each other.

A C₁-C₆₀ heteroaryl group as used herein may refer to a monovalent group having a heterocyclic aromatic system that has at least one heteroatom selected from N, O, P, and S as a ring-forming atom, and 1 to 60 carbon atoms. A C₁-C₆₀ heteroarylene group as used herein may refer to a divalent group having a heterocyclic aromatic system that has at least one heteroatom selected from N, O, P, and S as a ring-forming atom, and 1 to 60 carbon atoms. Non-limiting examples of the C₁-C₆₀ heteroaryl group include a pyridinyl group, a pyrimidinyl group, a pyrazinyl group, a pyridazinyl group, a triazinyl group, a quinolinyl group, and an isoquinolinyl group. When the C₁-C₆₀ heteroaryl group and the C₁-C₆₀ heteroarylene group each independently include two or more rings, the respective rings may be fused to each other or may be linked with each other.

A C₆-C₆₀ aryloxy group as used herein may refer to a monovalent group represented by —OA₁₀₂ (wherein A₁₀₂ is the C₆-C₆₀ aryl group), and a C₆-C₆₀ arylthio group as used herein may refer to a monovalent group represented by —SA₁₀₃ (wherein A₁₀₃ is the C₆-C₆₀ aryl group).

A monovalent non-aromatic condensed polycyclic group (as used herein may refer to a monovalent group that has two or more rings condensed to each other, has only carbon atoms as ring-forming atoms (for example, 8 to 60 carbon atoms), and is non-aromatic in the entire molecular structure. A non-limiting example of the monovalent non-aromatic condensed polycyclic group includes a fluorenyl group. A divalent non-aromatic condensed polycyclic group as used herein may refer to a divalent group having the same structure as the monovalent non-aromatic condensed polycyclic group.

A monovalent non-aromatic condensed heteropolycyclic group as used herein may refer to a monovalent group that has two or more rings condensed to each other, has at least one heteroatom selected from N, O, Si, P, and S as a ring-forming atom, in addition to carbon atoms (for example, 1 to 60 carbon atoms), and which is non-aromatic in the entire molecular structure. An example of the monovalent non-aromatic condensed heteropolycyclic group includes a carbazolyl group. A divalent non-aromatic condensed heteropolycyclic group as used herein may refer to a divalent group having the same structure as the monovalent non-aromatic condensed heteropolycyclic group.

In the formulae of the present specification, at least one substituent of the substituted C₁-C₆₀ alkyl group, the substituted C₂-C₆₀ alkenyl group, the substituted C₂-C₆₀ alkynyl group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₆-C₆₀ aryloxy group, the substituted C₆-C₆₀ arylthio group, the substituted C₁-C₆₀ heteroaryl group, the substituted monovalent non-aromatic condensed polycyclic group, and the substituted monovalent non-aromatic condensed heteropolycyclic group may be selected from:

deuterium, —CD₃, —CD₂H, —CDH₂, —F, —Cl, —Br, —I, a hydroxy group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, and a C₁-C₆₀ alkoxy group;

a C₁-C₆₀ alkyl group, a C₂-C₆₀ alkenyl group, a C₂-C₆₀ alkynyl group, and a C₁-C₆₀ alkoxy group, each substituted with at least one selected from deuterium, —CD₃, —CD₂H, —CDH₂, —F, —Cl, —Br, —I, a hydroxy group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₁₁)(Q₁₂)(Q₁₃), —N(Q₁₄)(Q₁₅), —B(Q₁₆)(Q₁₇);

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;

a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —CD₃, —CD₂H, —CDH₂, —F, —Cl, —Br, —I, a hydroxy group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₂₁)(Q₂₂)(Q₂₃), —N(Q₂₄)(Q₂₅), and —B(Q₂₆)(Q₂₇); and Si(Q₃₁)(Q₃₂)(Q₃₃), —N(Q₃₄)(Q₃₅), —B(Q₃₆)(Q₃₇),

wherein Q₁₁ to Q₁₇, Q₂₁ to Q₂₇, and Q₃₁ to Q₃₇ may each independently be selected from hydrogen, deuterium, —F, —Cl, —Br, —I, a hydroxy group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted monovalent non-aromatic condensed polycyclic group, and a substituted or unsubstituted monovalent non-aromatic condensed heteropolycyclic group.

When a group containing a specified number of carbon atoms is substituted with any of the groups listed in the preceding paragraph, the number of carbon atoms in the resulting “substituted” group is defined as the sum of the carbon atoms contained in the original (unsubstituted) group and the carbon atoms (if any) contained in the substituent. For example, when the term “substituted C₁-C₃₀ alkyl” refers to a C₁-C₃₀ alkyl group substituted with C₆-C₃₀ aryl group, the total number of carbon atoms in the resulting aryl substituted alkyl group is C₇-C₆₀.

The term “biphenyl group” as used herein may refer to a monovalent group in which two benzene rings are linked together via a single bond.

The term “terphenyl group” as used herein may refer to a monovalent group in which three benzene rings are linked together via a single bond.

One or more exemplary embodiments of the present disclosure will now be described in detail with reference to the following examples. However, these examples are only for illustrative purposes and are not intended to limit the scope of the one or more exemplary embodiments of the present disclosure.

### EXAMPLES

### Example 1

### (i) Synthesis of 11,11,12,12,13,13,14,14-octacyano-1,4,5,8-anthraquinotetramethane (OCNAQ)

11,11,12,12,13,13,14,14-octacyano-1,4,5,8-anthraquinotetramethane (OCNAQ) was synthesized according to the following pathway.

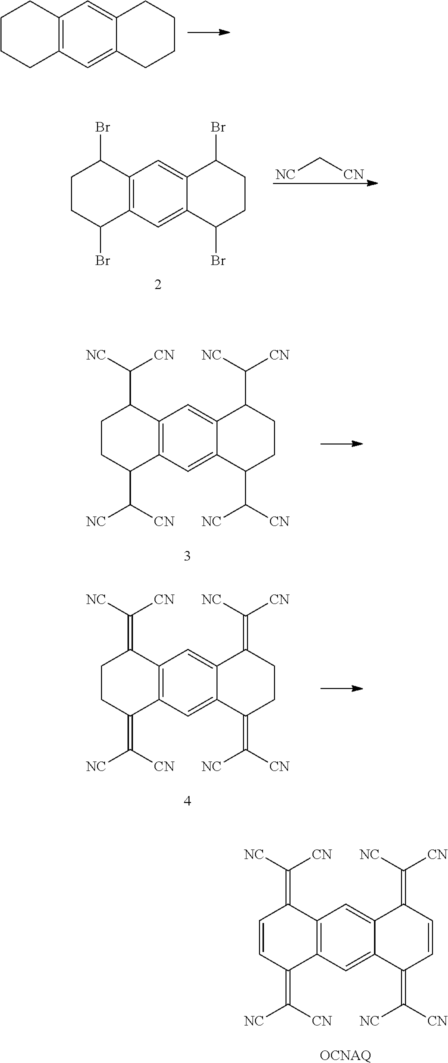

### Synthesis of 1,4,5,8-tetrabromo-1,2,3,4,5,6,7,8-octahydroanthracene (2)

3.00 grams (g) (16.1 millimoles, mmol) of 1,2,3,4,5,6,7,8-octahydroanthracene (1) and 150 milliliters (mL) of carbon tetrachloride were added into a 200-mL branched flask, and 12.6 g (70.2 mmol) of N-bromosuccinimide was added thereto under stirring. The resulting mixture was heated at about 95° C. for about 45 minutes under reflux, while being irradiated by a 100-W (100-watt) light bulb. After cooling, the resulting reaction solution was filtered, and the filtrate was washed with carbon tetrachloride, distilled water, ethanol, and then hexane to obtain 4.33 g of 1,4,5,8-tetrabromo-1,2,3,4,5,6,7,8-octahydroanthracene (2) as a white powder (yield: 54%).

Mp. 189-191° C., IR (KBr): v_{C—H}=2955 cm⁻¹, v_{C═C}=1498 cm⁻¹, v_{C—Br}629 cm⁻¹, ¹H NMR (CDCl₃): δ (ppm)=7.35 (s, 2H), 5 .56 (s, 4H).

### Synthesis of 1,4,5,8-tetrakis(dicyanomethyl)-1,2,3,4,5,6,7,8-octahydroanthracene (3)

8.00 g (200 mmol) of sodium hydride was added into a 300-mL, 3-necked flask. The flask was purged with nitrogen gas, and 24 mL of purified dimethyl sulfoxide (DMSO) was added thereto. A solution of 14 g (212 mmol) of malononitrile dissolved in 16 mL of purified DMSO was then added dropwise into the mixture for about 15 minutes while the mixture was stirred in a water bath, 4.32 g (8.6 mmol) of 1,4,5,8-tetrabromo-1,2,3,4,5,6,7,8-octahydroanthracene was then added thereto, and the resulting mixture was stirred at room temperature for about 3 hours. 200 mL of a 10% aqueous acetic acid solution was added to the resulting product maintained in the water bath, and the precipitate was filtered to obtain a brown, viscous solid. After washing this solid with distilled water, benzene, and then hexane and 40 mL of acetic acid were added thereto, and the mixture was stirred at about 80° C. for about 10 minutes. Then, 200 mL of distilled water was added thereto, and the mixture was stirred for about 5 minutes and filtered. The resulting filtrate was washed with distilled water, and then hexane, to obtain 2.32 g of 1,4,5,8-tetrakis(dicyanomethyl)-1,2,3,4,5,6,7,8-octahydroanthracene (3) as a white powder (yield: 61%).

Mp. 348° C., IR (KBr): v_{C—H}=2955 cm⁻¹, v_{C—N}=2259 cm⁻¹, v_{C═C}=1512 cm⁻¹, ¹H NMR (DMSO-d₆): δ (ppm)=7.53 (s, 2H), 5 .58 (d, J=5.0 Hz, 4H), 3.79 (br, 4H), 2.42-2.35 (m, 4H), 1.83-180 (m, 4H).

### Synthesis of 1,4,5,8-tetrakis(dicyanomethylene)-1,2,3,4,5,6,7,8-octahydroanthracene (4)

After treating the 300-mL, 3-necked flask with nitrogen, 24 mL of purified acetonitrile, 4 mL of acetic acid, 3.6 g (8.16 mmol) of 1,4,5,8-tetrakis(dicyanomethyl)-1,2,3,4,5,6,7,8-octahydroanthracene, and 8.00 g (44.8 mmol) of N-bromosuccinimide were added thereto. The resulting mixture was stirred at room temperature for about 1 hour, and then, at about 45° C. for about 2 hours. After cooling the resulting product in a water bath, a mixed solution of 20 mL of purified dimethylformamide (DMF), 1.2 mL of purified ethanol, and 10 mL of acetic acid was added dropwise thereto for about 1 hour, and the resulting mixture was stirred at room temperature for about 24 hours. Then, 40 mL of a 50% aqueous acetic acid solution was added thereto, and the mixture was stirred for about 1 hour, and then filtered. The resulting filtrate was washed with acetonitrile to obtain 2.6 g of 1,4,5,8-tetrakis(dicyanomethylene)-1,2,3,4,5,6,7,8-octahydroanthracene (4) as a yellow powder (yield: 57%).

Mp. 316° C., IR (KBr): v_{C—H}=3064 cm⁻¹ and 2914 cm⁻¹, v_{CN}=2227 cm⁻¹, v_{C═C}=1590 cm⁻¹.

### Synthesis of 11,11,12,12,13,13,14,14-octacyano-1,4,5,8-anthradiquino tetramethane (OCNAQ)

3.72 g (8.52 mmol) of 1,4,5,8-tetrakis(dicyanomethylene)-1,2,3,4,5,6,7,8-octahydroanthracene and 120 mL of purified acetonitrile were added into a 500-mL, 3-necked flask, and a mixed solution of 3.60 g (45.5 mmol) of pyridine and 60 mL of purified acetonitrile was added thereto and stirred for about 30 minutes. A mixed solution of 4.20 g (26.3 mmol) of bromine and 300 mL of a 50% aqueous acetic acid solution, cooled in an ice bath, was added to the resulting product, and the mixture was stirred in a water bath for about 10 minutes. The resulting reaction system was filtered, and the filtrate was washed with a 50% aqueous acetic acid solution, distilled water, ethanol, and then, acetonitrile to obtain 2.3 g of 11,11,12,12,13,13,14,14-octacyano-1,4,5,8-anthradiquinotetramethane (OCNAQ) as a yellow powder (yield: 68%).

Mp. >400° C., IR (KBr): v_{C—H}=3064 cm⁻¹, v_{cN}=2223 cm⁻¹, v_{C═}=1598 cm⁻¹.

### (ii) Manufacture of Secondary Battery

After 200 mg of OCNAQ, 200 mg of carbon black, and 21 mg of polytetrafluoroethylene (PTFE) were mixed in a mortar, a sheet having at a thickness of about 180 pm was formed using the resulting mixture, and then, pressed at φ9 millimeters (mm) to form a positive active material layer. A solution of 0.8 moles per liter (mol/L) of LiN(CF₃SO₂)₂ dissolved in a mixed solution of 1,2-dimethoxyethane and 1,1,2,2-tetrafluoroethyl-2,2,3,3-tetrafluoropropyl ether at a volume ratio of about 1:4 was used as an electrolyte solution. A glass fiber separator, which was impregnated with 60 microliters (μl) of the electrolyte solution, and the positive active material layer were stacked on lithium metal (φ15 mm and a thickness of about 100 μm) to manufacture a CRS-2032-type coin cell. Every battery manufacturing process was performed in an argon (Ar) glove box.

### (iii) Electrochemical Evaluation

A charge and discharge test was performed on the manufactured battery at about 25° C. with a cutoff potential of about 1.8 volts (V) to about 3.8 V. After repeated charge and discharge cycles at 0.1 C, capacity retention with respect to the number of cycles was evaluated.

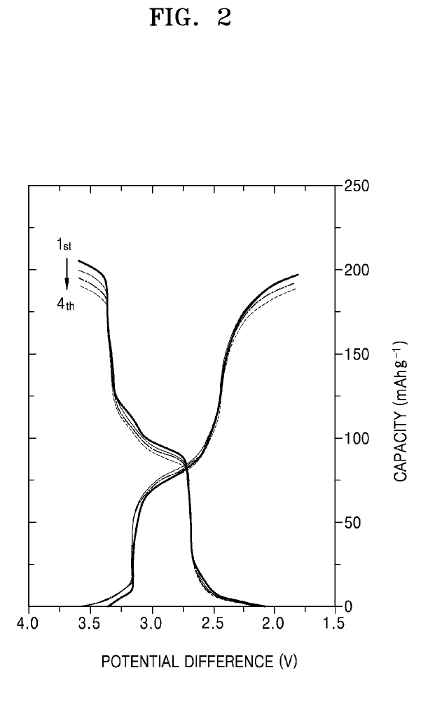

FIG. 2 is a charge and discharge curve of a secondary battery of Example 1. Referring to FIG. 2, the secondary battery of Example 1 including OCNAQ as a positive active material had a high capacity of about 200 milliampere hours per gram (mAhg⁻¹) and sufficient capacity retention after four charge and discharge cycles. Referring to FIG. 2, the secondary battery of Example 1 was found to have oxidation-reaction potentials (ORPs) at around 2.5 V and 3.25 V.

### Comparative Example 1

A secondary battery was manufactured and evaluated under the same conditions as those of Example 1, except that the positive active material used in Example 1 was replaced with tetracyanoquinodimethane (TCNQ).

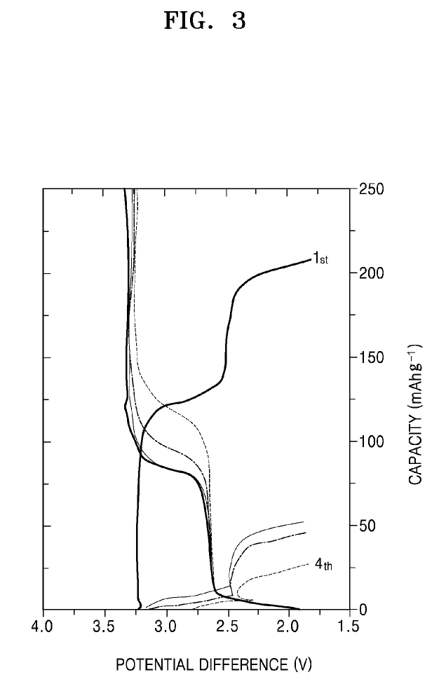

FIG. 3 is a charge and discharge curve of the secondary battery of Comparative Example 1. Referring to FIG. 3, the secondary battery of Comparative Example 1 including TCNQ as the positive active material was found to have a significantly reduced capacity after 2^{nd} to 4^{th} charge and discharge cycles, as compared with that after a 1^{st} cycle. As shown in FIG. 3, it appears that charging of the secondary battery of Comparative Example 1 was continuous even with such significant reduction in discharge capacity. This seems to be attributed to the so-called redox shuttle phenomenon in which the dissolved TCNQ (positive active material) transports charge repeatedly between the positive electrode and the negative electrode at around 3.3 V.

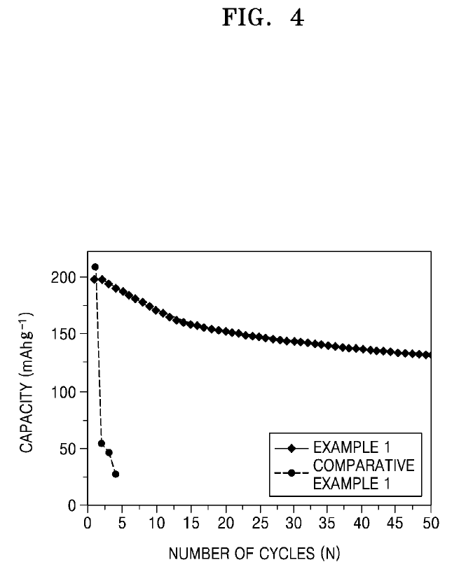

FIG. 4 is a graph of capacity retention with respect to numbers of cycles of the secondary batteries of Example 1 and Comparative Example 1. Referring to FIG. 4, the secondary battery of Comparative Example 1 was found to have a sharp reduction in capacity with an increasing number of cycles, with capacity after the 4^{th} cycle reduced to 12.9% of the initial capacity. However, the secondary battery of Example 1 was found to exhibit sufficiently suppressed reduction in capacity even after the 50^{th} cycle, and in particularly, with a capacity retention of about 95.9% at the 4^{th} cycle.

As described above, the secondary battery according to an embodiment was found to have high capacity and good cycle characteristics.

As described above, according to the one or more embodiments, a secondary battery with improved capacity and improved cycle characteristics may be manufactured using an electrode active material according to any of the above-described embodiments.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the present disclosure as defined by the following claims.

What is claimed is:

1. An electrode active material comprising:
at least one selected from a quinodimethane compound and a lithium salt thereof, wherein the quinodimethane compound comprises at least three linearly-condensed aryl groups and at least two quinodimethane moieties.

2. The electrode active material of claim 1, wherein the quinodimethane compound is a compound represented by Formula 1: 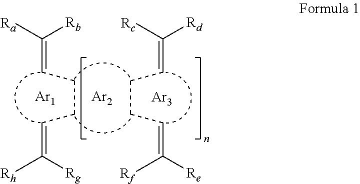
wherein, in Formula 1,
Ar₁ to Arₐ are each independently a substituted or unsubstituted C₅-C₆₀ carbocyclic group, or a substituted or unsubstituted C₁-C₆₀ heterocyclic group;
Rₐ to Rₕ are each independently selected from a hydrogen atom, a halogen atom, a hydroxyl group, a nitro group, a cyano group, a carboxylic acid group, a substituted or unsubstituted C₁-C₆₀ alkyl group, a substituted or unsubstituted C₆-C₆₀ aryl group, and a substituted or unsubstituted C₁-C₆₀ heteroaryl group;
n is an integer of 1 or greater;
at least one substituent of each of the substituted C₅-C₆₀ carbocyclic group and the substituted C₁-C₆₀ heterocyclic group is selected from deuterium (-D), —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, and a C₁-C₆₀ alkoxy group;
a C₁-C₆₀ alkyl group, a C₂-C₆₀ alkenyl group, a C₂-C₆₀ alkynyl group, and a C₁-C₆₀ alkoxy group, each substituted with at least one selected from deuterium, —F, —Cl, —Br, —I, a hydroxyl group, a cyano group, a nitro group, an amidino group, a hydrazino group, a hydrazono group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₁₁)(Q₁₂)(Q₁₃), —N(Q₁₁)(Q₁₂), -B(Q₁₁)(Q₁₂), —C(═O)(Q₁₁), —S(═O)₂(Q₁₁), and —P(═O)(Q₁₁)(Q₁₂);
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, and a monovalent non-aromatic condensed heteropolycyclic group, each substituted with at least one selected from deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₆-C₆₀ aryloxy group, a C₆-C₆₀ arylthio group, a C₁-C₆₀ heteroaryl group, a monovalent non-aromatic condensed polycyclic group, a monovalent non-aromatic condensed heteropolycyclic group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), and —P(═O)(_{Q21})(_{Q22}), and
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), and —P(═O)(Q₃₁)(Q₃₂),
wherein Q₁₁ to Q₁₃, Q₂₁ to Q₂₃, and Q₃₁ to Q₃₃ are each independently selected from hydrogen, deuterium, —F, —Cl, —Br, —I, a hydroxyl group, a cyano group, a nitro group, an amidino group, a hydrazino group, a hydrazono group, a C₁-C₆₀ alkyl group, a C₂-C₆₀ alkenyl group, a C₂-C₆₀ alkynyl group, a C₁-C₆₀ alkoxy group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, a monovalent non-aromatic condensed heteropolycyclic group, a biphenyl group, and a terphenyl group.

3. The electrode active material of claim 2, wherein at least one selected from Rₐ to Rₕ is a cyano group.

4. The electrode active material of claim 2, wherein Ar₁ to Ar₃ are each independently selected from
a benzene group, a naphthalene group, a phenalene group, a phenanthrene group, an anthracene group, a fluoranthene group, a triphenylene group, a pyrene group, a chrysene group, a naphthacene group, a picene group, a perylene group, and a pentaphene group; and
a benzene group, a naphthalene group, a phenalene group, a phenanthrene group, an anthracene group, a fluoranthene group, a triphenylene group, a pyrene group, a chrysene group, naphthacene group, a picene group, a perylene group, and a pentaphene group, each substituted with at least one selected from deuterium, —F, —Cl, —Br, —I, a hydroxyl group, a cyano group, a nitro group, an amidino group, a hydrazino group, a hydrazono group, a C₁-C₂₀ alkyl group, a C₁-C₂₀ alkoxy group, a phenyl group, a biphenyl group, a terphenyl group, a naphthyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), and —P(═O)(Q₃₁)(Q₃₂),
wherein Q₃₁ to Q₃₃ are each independently selected from a C₁-C₁₀ alkyl group, a C₁-C₁₀ alkoxy group, a phenyl group, a biphenyl group, a terphenyl group, and a naphthyl group.

5. The electrode active material of claim 2, wherein the quinodimethane compound is a compound represented by Formula 1A or 1B: 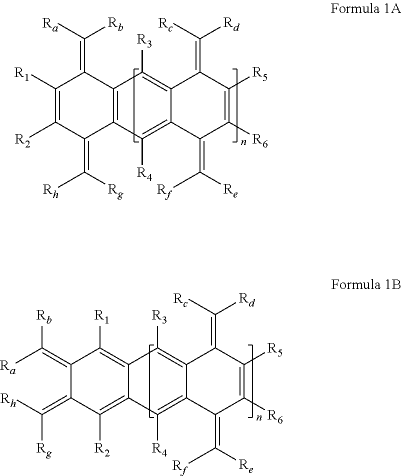
wherein, in Formulae 1A and 1B,
Rₐ to Rₕ are each independently selected from a hydrogen atom, a halogen atom, a hydroxyl group, a nitro group, a cyano group, a carboxylic acid group, a substituted or unsubstituted C₁-C₆₀ alkyl group, a substituted or unsubstituted C₆-C₆₀ aryl group, and a substituted or unsubstituted C₁-C₆₀ heteroaryl group,
at least one selected from Rₐ to Rₕ is a cyano group,
R₁ to R₆ are each independently selected from a hydrogen atom, a halogen atom, a hydroxyl group, a nitro group, a cyano group, a carboxylic acid group, a substituted or unsubstituted C₁-C₆₀ alkyl group, a substituted or unsubstituted C₆-C₆₀ aryl group, and a substituted or unsubstituted C₁-C₆₀ heteroaryl group, and n is an integer of 1 or greater.

6. The electrode active material of claim 2, wherein the quinodimethane compound is a compound represented by Formula 1A-1: 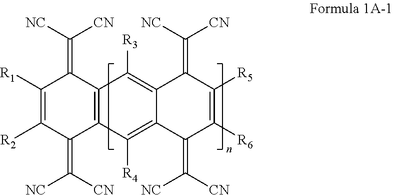
wherein, in Formula 1A-1, R₁ to R₆ are each independently selected from a hydrogen atom, a halogen atom, a hydroxyl group, a nitro group, a cyano group, a carboxylic acid group, a substituted or unsubstituted C₁-C₆₀ alkyl group, a substituted or unsubstituted C₆-C₆₀ aryl group, and a substituted or unsubstituted C₁-C₆₀ heteroaryl group, and
n is an integer of 1 or greater.

7. The electrode active material of claim 6, wherein n is an integer selected from 1 to 4.

8. The electrode active material of claim 6, wherein n is 1.

9. The electrode active material of claim 6, wherein each of R₁ to R₆ is a hydrogen atom.

10. The electrode active material of claim 1, wherein the electrode active material comprises about 60 percent by weight or more of the quinodimethane compound based on a total weight of the electrode active material.

11. The electrode active material of claim 1, wherein the electrode active material comprises about 90 percent by weight or more of the quinodimethane compound based on a total weight of the electrode active material.

12. The electrode active material of claim 1, wherein the quinodimethane compound has two or more oxidation-reduction potentials in a range of about 1.0 volts to about 4.0 volts with respect to Li/Li⁺.

13. The electrode active material of claim 12, wherein the quinodimethane compound has two or more oxidation-reduction potentials in a range of about 2.0 volts to about 3.5 volts with respect to Li/Li⁺.

14. The electrode active material of claim 1, wherein the electrode active material is a positive active material.

15. A secondary battery comprising:
a positive electrode comprising an electrode active material of claim 1;
a negative electrode; and
an electrolyte membrane.

16. The secondary battery of claim 15, wherein the negative electrode comprises at least one selected from a graphite active material, a silicon oxide or an alloy thereof, a tin oxide or an alloy thereof, a titanium oxide compound, and an alkali metal.

17. The secondary battery of claim 15, wherein the electrolyte membrane comprises a solid electrolyte or a polymer electrolyte.

18. The secondary battery of claim 17, wherein the solid electrolyte is a phosphoric acid solid electrolyte.

19. The secondary battery of claim 15, wherein the secondary battery has a cutoff potential of about 1.8 volts to about 3.8 volts, and a discharge capacity of about 150 milliampere hours per gram or greater after 2 to 4 charge and discharge cycles at about 25° C.

20. The secondary battery of claim 15, wherein the secondary battery has a cutoff potential of about 1.8 volts to about 3.8 volts, and capacity retention of about 80% or greater after 50 charge and discharge cycles at about 25° C.
